(19) United States
(12) Patent Application Publication
(10) Pub. No.: US 2008/0274512 A1
(43) Pub. Date: Nov. 6, 2008

(54) Reagent Transfer Device

(76) Inventors: David James Squirrell, Wiltshire (GB); Georgina Martin, Saxmundham (GB)

Correspondence Address: JOHN S. PRATT, ESQ;KILPATRICK STOCKTON, LLP, 1100 PEACHTREE STREET, ATLANTA, GA, 30309, US

(21) Appl. No.: 11/884,801

(22) PCT Filed: Feb. 27, 2006
(86) PCT No.: PCT/GB2006/000689

§ 371 Date: Jul. 1, 2008

(30) Foreign Application Priority Data
Feb. 26, 2005 (GB) 0503986.2

(51) Int. Cl. C12P 19/34; B01L 11/00; G01N 1/00

(52) U.S. Cl. 435/91.2; 422/99; 436/174

(57) ABSTRACT

A device for transferring reagents in solid or liquid form from one place to another on an apparatus, said device comprising an upper portion, adapted to be held, and a lower portion adapted to hold reagents in solid or liquid form, in an available manner. For example, the reagents may be held within a cage structure or retained on an outer surface of the lower portion. Methods for using the device, as well as apparatus for use in conjunction with the device are also described and claimed.

The present invention relates to devices for moving reagents used in chemical and biochemical assays, as well as methods using these devices.

There is frequently a need to transfer small quantities of reagents, in solid form or liquid form, from one vessel to another in the course of conducting chemical or biochemical assays. Increasingly, these assays are conducted using automated processes and procedures and so automated devices, capable of picking up and transferring quantities of reagents are required.

Particular examples of reagents, which are available in solid form, are the reagents necessary for carrying out an amplification reaction, in particular the Polymerase Chain Reaction or PCR. Commercial PCR ready-to-go beads are available for carrying out DNA amplification, such as those sold by Amersham BioSciences UK.

They contain all the components necessary to carry out standard PCR including buffer reagents, salts and polymerase enzymes, in a freeze-dried or other solid form. They therefore provide a convenient means of providing stabilised reagents for end users to assemble into assay cartridges with, for example “homebrew” primers and probes. They benefit from mass production and a reproducible formulation in a convenient storage format that has a long shelf life.

However, generally beads require tweezers to transfer from one consumable to another. This can be difficult to achieve efficiently, in particular in automated devices.

Liquids are usually transferred using pipettes or similar. These also can be relatively difficult to use, and liable to drip during a transfer procedure, in particular where this is effected automatically.

The applicants have developed a device for the efficient manipulation of reagents.

According to the present invention, there is provided a device for transferring reagents in solid or liquid form from one place to another on an apparatus, said device comprising an upper portion, adapted to be held, and a lower portion adapted to hold reagents in solid or liquid form, externally in an available manner.

As used herein, the expression “externally in an available manner” means that the reagents are in contact with the surrounding environment. For instance, they are located on an outer surface of the lower portion, or are otherwise available such that, for example, immersion of the said lower portion in a liquid releases reagents held thereon into said liquid. In accordance with the invention, the lower portion includes a particular adaptation which makes facilitates the accommodation of liquid or solid samples in this manner.

In a preferred embodiment, the lower portion of the device comprises a detachable open cage structure, adapted to hold solid or liquid reagents, and in particular solid reagents.

The term “solid reagent” as used herein refers to one or more agents or chemicals which are in solid form. For instance, they may comprise powders, crystals, granules or beads. In particular, they comprise a combination of reagents, which are combined together in a bead form, such as the PCR ready-to-go beads, as described above. Where reagents are generally found in a liquid form such as in solution in water, suitable solid forms may be prepared by conventional methods such as freeze-drying or spray drying. The beads or granules may further comprise conventional agents such as fillers, dispersants, surfactants etc. to ensure that the granules or beads dissolve or disperse when immersed in liquids such as water.

The cage structure suitably comprises a base portion, which in particular when it is intended to carry small quantities of liquid reagents, has solid base and sidewalls. However, above this portion, one or more apertures are provided in the side walls. Where the cage structure is intended to carry solid reagents, for example in the form of large reagent beads, the base may also have one or more apertures therein. Any apertures should be of a size that does not allow solid reagents, for example reagent beads contained within the cage structure, to pass out of the cage structure. However, these apertures allow liquid to enter cage structure, when the lower portion is immersed, whereupon reagents contained in the cage structure disperse or dissolve in the liquid.

The cage structure is suitably made of a plastics material, which is inert in the reaction mixture in which it is required to be immersed. It is detachable from the upper portion of the device for filling purposes. Therefore it is suitably arranged to fix to the upper portion by means of a snap-fit or screw-threaded arrangement.

Cage structures as described above, form a further aspect of the invention.

In an alternative embodiment, the lower portion comprises a wand, which is able to hold reagents in dried form deposited on an outer surface thereof. Reagents in dried form are suitably either freeze-dried or spray-dried, but are preferably freeze-dried.

As used herein, the term “wand” refers to any suitable structure, which may be introduced into and moved between reaction vessels and the like. Generally it will be elongate in shape, for example a rod-like tubular structure, although the sides may be inclined, so as to form a syringe-like or conical profile.

It may be solid or hollow in nature. Where the wand is hollow, it may be able to accommodate additional elements, such as magnets. In this case, introduction of a magnet into a wand may allow it to be used also for the collection of magnetic solids, such as magnetic beads, like magnetic silica beads, which optionally carry further reagents such as binding reagents like antibodies or binding fragments thereof.

Suitably the outer surface of the wand is profiled to allow reagents to be accommodated within the profiled regions. These profiled regions may comprise dimples or grooves, which increase the surface area and so allow for more reagent to be freeze-dried thereon. They may also be structured to facilitate the separate drying on of different reagents in different locations from one another. This may be particularly useful where different components of a mixture may be incompatible in freeze-drying or long-term storage.

The reagents are suitably deposited onto the outer surface of the wand using conventional drying and in particular freeze-drying techniques. For example, a mixture of the reagents, in solution or dispersion in a suitable solvent, is formed, and the wand immersed into this. Then solvent is removed by freeze-drying, leaving a coating of reagents on the surface of the wand. Provided the wand is not subject to significant mechanical abrasion, this reagent coating will remain in place, until the rod is immersed in a suitable solvent or solution, whereupon the reagents will dissolve or become dispersed in the liquid.

The size of these grooves or dimples will depend upon the size of the wand and the apparatus in which it is to be used. Generally however, any dimples will be from 0.5-2 mm diameter and depth, and similarly grooves will be from 0.5 to 2 mm wide and deep.

Any profiling is suitably arranged on a lower surface of the wand.

In a particular embodiment, the lower surface of the wand may be provided with an expanded carrier portion, which may be shaped profiled for the optimal transfer of liquid or solid reagents. For example, the expanded portion may have a substantially hollow or tubular shape, optionally with slits in the sidewalls thereof. Immersion of the expanded portion into a liquid reagent in a first container will result in the retention of liquid within the expanded portion, at least temporarily, due to surface tension. If the device is then moved as required, the liquid can be dispensed into an appropriate second container, either by immersion in a liquid within that second container, or by gentle shaking of the device.

The expanded portion may also carry dried solid reagents which may be loaded as described above.

The wand may be integral with the upper portion, or it may be detachable therefrom as a result of for example, a snap-fit or screw thread arrangement, as described above.

Suitably the device is at least partially disposable after use. In particular, the wand is disposable.

Wands having dried reagents on an outer surface thereof, form a further aspect of the invention.

The upper portion of the device is suitably adapted to be detachably held within a transfer means of an assay apparatus. The shape of the upper portion will therefore be variable depending upon the assay apparatus being used. In a particular example however, the upper portion may be provided with one or more annular flanges, projections or ridges, which are arranged to interact with suitable grabber means on an assay apparatus.

Alternatively, the upper portion may comprise a magnet or a ferromagnetic element, allowing it to be held and moved using magnetic transfer means. Such means may, for example comprise a further hollow wand which can have magnet removably inserted therein as described above.

For instance the upper portion may be adapted to hold or an insert of for example soft iron, or is moulded from plastic comprising for example polypropylene blended with iron filings.

Suitably, the upper portion in this case comprises a “goblet”-shaped reagent carrier where the bowl of the goblet either contains an insert of for example soft iron inserted into the base of the goblet, or is moulded from plastic comprising for example polypropylene blended with iron filings. This allows the goblet to be picked up magnetically, for example using a magnetic wand as described above, which can be is inserted into the “bowl” of the goblet, which allows it then to be lifted up, and released by the magnets withdrawal.

Suitably, a complementary loading apparatus is provided. This apparatus is able to load dried reagents onto the lower portions of the device. This suitably comprises a body, for example of a freeze drying block provided with holes into which one or more of the devices of the invention may be inserted, such that the lower portion projects out of the body, and into a sealable chamber. Suitably, the devices are inverted for this purpose, so that the lower portion projects upwardly into the chamber.

Reagent or reagent mixtures in liquid form are then added to the chamber and subjected to, for example, freeze drying, which means that they become deposited on the lower portion.

During this process body is suitably clamped between two plates so as to ensure that the one or more devices remain in position. The upper clamp suitably forms an upper surface of the chamber, and suitably is provided with a sealable opening to allow reagents to be added.

The loading apparatus, or at least the plates thereof, are suitably made of a thermally conducting material able to withstand the conditions encountered during freeze-drying, such as a metal, for example brass, or a plastic that can be moulded but has good thermal conductivity e.g. commercially available cool polymer.

Loading apparatus of this type, in particular in combination with a device as described above, forms a further aspect of the invention.

The invention further provides a method for delivering reagents into a liquid in a reaction vessel, said method comprising introducing a device as described above holding reagents on the lower portion thereof, into the reaction vessel, and releasing said reagents into the reaction vessel.

In the case of liquid reagents, these may be released by shaking the device so that surface tension holding the liquid in place is overcome. Alternatively, or in the case of solid reagents, release is suitably achieved by at least partially immersing the lower portion of the device into a liquid within the reaction vessel, and allowing the reagents to dissolve or disperse into said liquid.

The liquid is a suitably any solvent or solution required for the next stage of the assay. For instance, in the case of reagents comprising PCR reagents such as a PCR bead, this may be dispensed by submerging the device into the resuspension buffer. In other cases, one might want to add the reagents to a DNA/RNA extract.

The method is extremely useful in that it allows the efficient transfer of reagents. Furthermore, it is particularly amenable to automation and may be included in a wide range of assay devices.

In a particularly preferred embodiment of the invention, the device further comprises reagents loaded onto said lower portion.

The reagents may be pre-loaded into the device using any suitable conventional method. The loading of dried reagents onto a wand has been described hereinbefore. Reagents such as PCR beads may be added to a cage structure as described above using tweezers or the like, before the cage structure is attached to the upper portion of the device. Liquids may be introduced using pipettes or the like, but these operations may be carried out away from the assay apparatus, thus avoiding any soiling of the apparatus itself.

Suitably, the device is then moved so as to release the reagents into the required reaction vessel, but the reaction vessel may be moved towards the device, in any way suited to the particular assay and assay apparatus being used.

This operation is suitably carried out automatically. This may for example involve movement of the device vertically upwards, for example to remove it completely from a first container, and then horizontally so that it is aligned with a second container, and then downwards so that the lower portion of the device is immersed in a liquid within the second container. Alternatively, after removal of the device from a first container by for instance an upwards movement of the device, the containers themselves may be moved so that the second container is arranged below the device. The device may then simply be lowered so that it enters the second container as necessary.

Suitable transport means are well known in the art, and may comprise conveyor belts, carousels or the like.

Suitable containers may be any reaction vessel, including individual reaction vessels or reaction wells in plates or the like, and the transport means will be adapted to move these if necessary.

A particular automated assay apparatus, which may benefit from the use of a device of the present invention is described and claimed in co-pending International Patent Application No. PCT/GB04/003363, the content of which is incorporated herein by reference. The apparatus described here is able to carry out highly complex multi step processing of samples. The apparatus comprises:
(i) a platform comprising:
   (a) a chamber suitable for receiving a sample; and
   (b) a functional component;
(ii) an arm capable of being raised and lowered and including a means for removeably attaching to the functional component such that said component may be raised and lowered with the arm; and
(iii) a means for moving the platform such that any chamber or functional component may be aligned with respect to the arm.

This apparatus has a wide variety of applications, and can be adapted for a wide variety of uses. The term “functional component” is defined as meaning an element of the apparatus that has been designed such that it can reversibly attach to the arm of the apparatus. The functional component can be designed to have a wide variety of uses as will be apparent from the disclosure herein. The specific use of one or more functional components can be readily identified by one skilled in the art depending on the specific use of the apparatus. For example the functional component may comprise a means for interacting with the fluid sample. Such a means may provide some physical processing to the sample for example heating, cooling, optics, sonication, and the like. Alternatively the functional component may comprise a means for interacting with the chamber itself, for example by acting as a cutter to pierce a foil seal, to cap the chamber, to introduce a filter and the like. Furthermore the functional component may act as a collector for moving the sample, or an analyte contained therein to another chamber of the apparatus.

In this context therefore, the device of the present invention may comprise a specific functional component of the apparatus of International Patent Application No. PCT/GB04/003363. In particular, it will comprise a component which is suitable for transferring PCR reagents into a reaction vessel, for use in an automated PCR process.

In a particular embodiment of the device, the platform is essentially circular and moves by rotation. This allows the platform to align the chambers or functional components with respect to the arm or other physical means. This also has the advantage of minimising the size of the apparatus when several different components are involved. Optionally the platform can be fitted with a sensing mechanism to allow for correct positioning of the functional component or chamber as the platform moves under the arm or other physical processing means located above the platform. However, it is further advantageous in that it allows for the easy centrifugation of a reaction vessel held on the platform.

In a preferred embodiment of the device of International Patent Application No. PCT/GB04/003363, the functional component comprises a sheath, which provides an interface between a magnet for attracting magnetic reagent beads, which may have immobilised thereon analytes or reactants, and the beads themselves. Preferably the sheath is located on the platform and is made of a material such that when the magnet is inside the sheath the complex will be attracted to the sheath. In such an embodiment it is preferred that the apparatus comprise a magnet co-located with the arm of the apparatus that can be lowered into the sheath to apply a magnetic field and raised out of the sheath to remove the magnetic field. The sheath is then placed into a chamber of the apparatus comprising the magnetic reagent beads. The magnet is lowered into the sheath and the magnetic reagent beads binds to the sheath. The sheath and magnet are then raised. The platform moves such that a new chamber is aligned, the sheath and magnet are then lowered and the magnet removed. When the magnet is removed the magnetic reagent beads will fall away from the sheath into the second chamber. Small movements of the sheath up and down by the arm will ensure that no magnetic reagent beads remains bound to the sheath and will also act to mix the magnetic reagent beads with any reagents or solutions in the new chamber. Alternatively an analyte can be eluted from the beads by any suitable means.

The magnet and the arm are designed to interact with each other without affecting the operation of the other such that the sheath can be independently raised and lowered with or without the magnet in place.

In a particular embodiment, if the sheath is formed as a wand or goblet-shaped embodiment as discussed above, it may also be used to transfer solid reagents in accordance with the present invention.

Apparatus for carrying out assays which incorporate the device described above form a further aspect of the invention.

In particular therefore, the invention further provides apparatus comprising a device as described above, and means for transferring said device from a first container to a second container.

The invention will now be particularly described by way of example with reference to the accompanying diagrammatic drawings, in which:

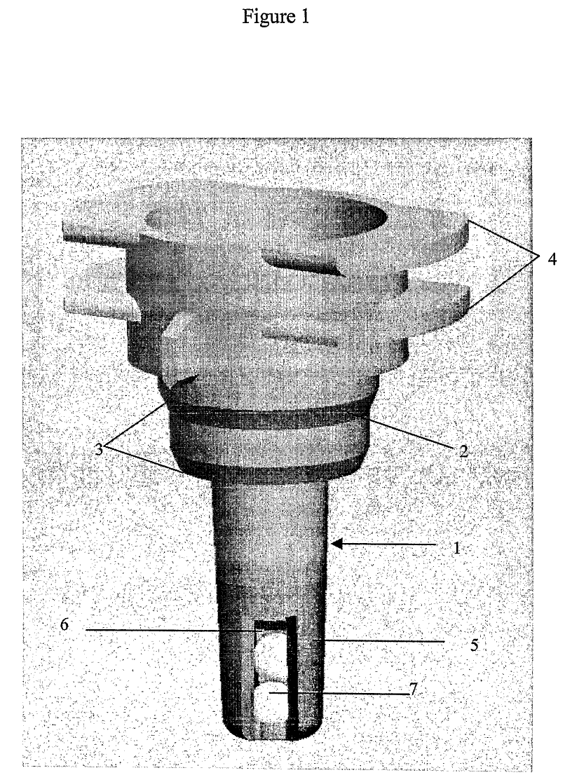

FIG. 1 is a side view of a first embodiment of a device of the invention;

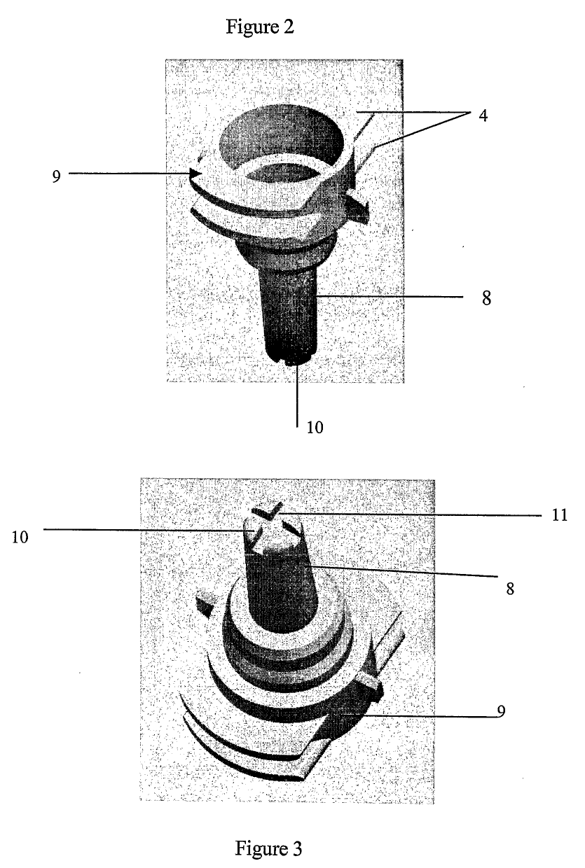

FIG. 2 shows a perspective top view of a second embodiment of a device of the invention;

FIG. 3 shows a perspective bottom view of the wand of FIG. 2;

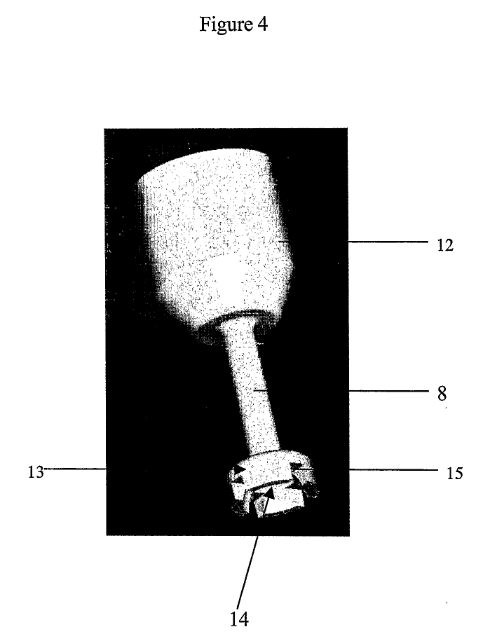

FIG. 4 shows a perspective side view of a third embodiment of a device of the invention;

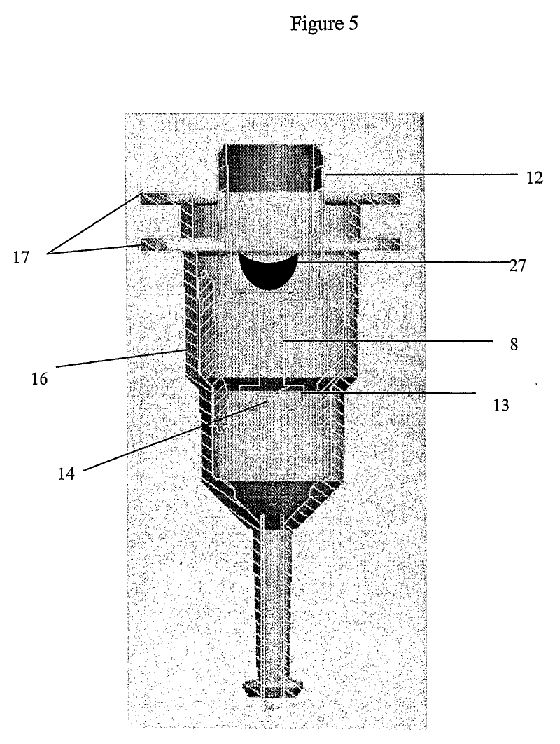

FIG. 5 shows a side section through apparatus containing the devices of FIG. 4,

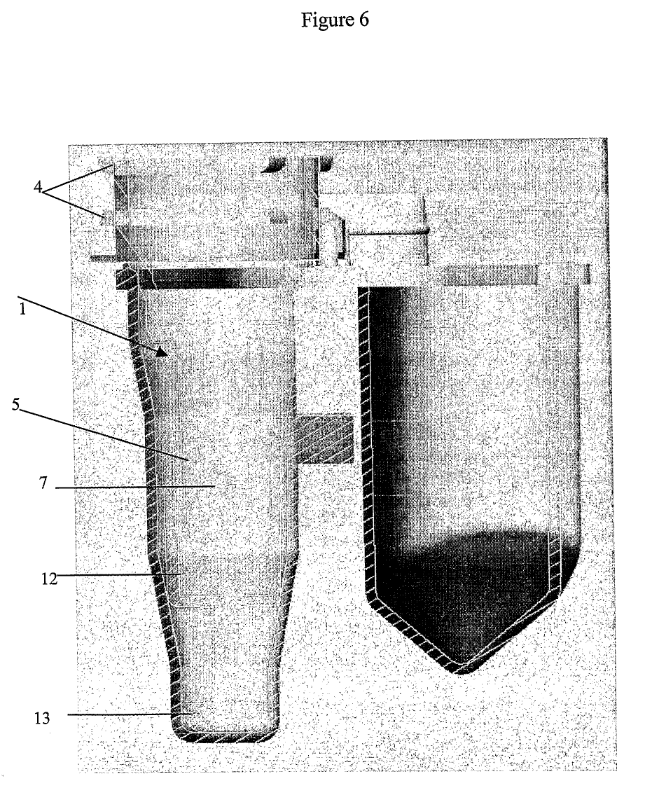

FIG. 6 shows a side section of a similar apparatus containing the devices of FIGS. 1 and 4;

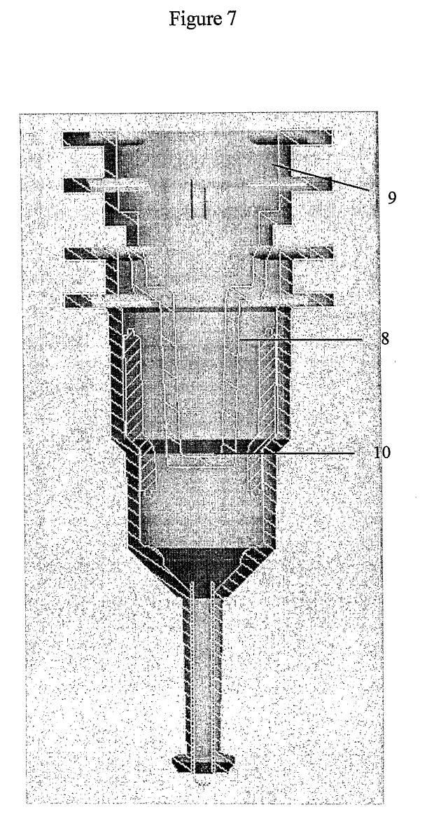

FIG. 7 shows a side section of a similar apparatus containing the device of FIGS. 2 and 3;

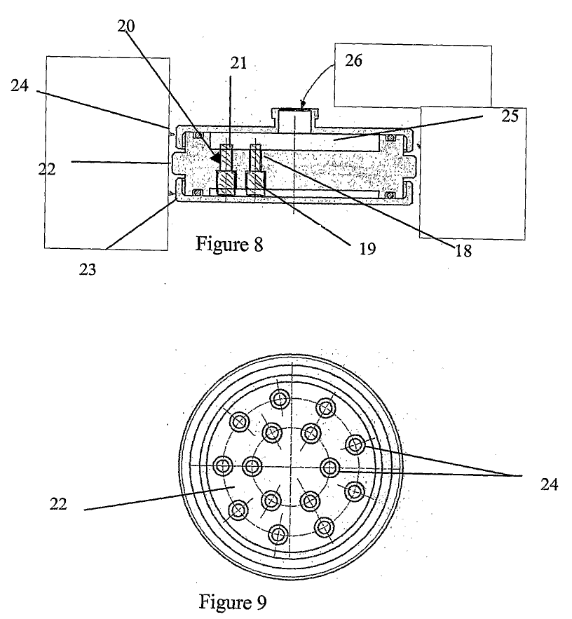

FIG. 8 shows a side section through a complementary loading device; and

FIG. 9 shows a top view of the device of FIG. 8, with a top cover removed to illustrate the arrangement of the holes.

FIG. 1 illustrates a device (1) comprising an upper portion (2) including a range of ridges (3) and projections (4) which adapt it to be detachably held by a grabber arm of an assay apparatus (not shown). A lower portion (5) is snap-fitted onto the base of the upper portion (2).

The lower portion (5) is provided with a number of vertically arranged apertures (6), which allow access for liquid to reagent beads (7) contained therein.

The reagent beads (7) were added to the lower portion (5) before it was fitted to the upper portion (2). The apertures are of such as size that the reagents beads (7) cannot fall out of the lower portion (2) during movement of the device.

Therefore, it is possible to transfer the beads (7) conveniently and securely from one reagent container to another within an apparatus, but attaching the upper portion (2) to the grabber arm of an assay apparatus, and using this to move the device as required.

When the lower portion (5) is immersed in a liquid in a reaction vessel for example, in a PCR resuspension buffer containing the sample, the beads (7) dissolve. Thereafter, the assay can be continued as required. If required, after dissolution of the bead, the device (1) can be removed from the reaction vessel, for instance using the transfer means of the apparatus.

An alternative embodiment of the invention is illustrated in FIG. 2. The wand of this embodiment has an elongate shaft (8). The upper portion (9) is provided with a pair of annular flanges (4), which allows it to be attached to a device for automatically moving it from one position to another.

A lower surface (10) of the shaft (8) is provided with a cruciate groove (11) (FIG. 3) in which freeze dried reagents can be accommodated.

Simple immersion of the lower surface (10) of the wand will result in the dissolution of the reagents from the groove (11).

Therefore, the invention provides a convenient and secure means of transferring reagents from one container to another.

An alternative embodiment is illustrated in FIG. 4. In this embodiment, the upper portion (12) is shaped as an open cup or goblet, allowing the introduction of transfer means.

The wand (8) in this case is provided with an expanded portion 13, which has a hollowed out base (14) and a number of annularly spaced grooves (15). This design is particularly useful for collecting liquid reagents and holding them temporarily in an available manner by surface tension.

The upper portion (12) has a soft iron insert (27) wedged therein (see FIG. 5). This allows the device to be moved using for example a magnetic or a magnetic wand as is known in the art. In the embodiment illustrated in FIG. 5, the device has been deposited in a reaction vessel (16) for use in the apparatus of International Patent Application No. PCT/GB04/003363. This reaction vessel itself has flanges (17) allowing it to be moved using a grabber arm.

FIGS. 6 and 7 illustrate how the devices of other embodiments of the invention may also be included in such apparatus. For instance, in FIG. 6, shows apparatus where a device as shown in FIG. 1 is stacked on top of a goblet type device, as illustrated generally in FIG. 4. FIG. 7 shows that the embodiment of FIGS. 2 and 3 can be similarly accommodated in a reaction vessel.

These combinations provide a huge amount of flexibility allowing many different types of assay and reaction to be conducted in the apparatus.

FIG. 8 shows a schematic side section of a reagent preparation or reagent loading apparatus, adapted specifically for the loading of solid reagents on a device of the invention. In this instance, the device (20) comprises a plastics wand (18) having a goblet shaped upper portion (19), which accommodates a small magnet such as an iron slug, or it may be made of an iron-filing filled polymer, allowing it to be picked up easily, magnetically.

The lower surface (21) of the lower portion of the device (20) is provided with a cruciate groove, similar to that provided in the device of FIGS. 2 and 3.

The loading apparatus itself comprises a freeze-drying block (22), with holes (24) (FIG. 9) therein, each of which is shaped to accommodate a device (20) so that the lower surface (21) protrudes out of the block (22). When the devices (20) are in place in the block (22), they are secured in position by means of a first clamp plate (23), which is screwed onto the block (22). The block (22) is then inverted and liquid reagents may be dispensed onto the lower surface (21) and thereby into the cruciate groove. A second or upper clamp plate (24) is then secured to the block (22) by means of screws. The upper plate (24) is designed so that a chamber (25) is defined between it and the upper surface of the block (22) into which the lower surface (21) protrudes). The upper plate (24) is also provided with a hole (26), which is closeable using a stopper, such as a rubber bung (not shown).

Rubber gaskets (not shown) are suitably provided to seal the plates (23, 24) to the body (22).

After insertion of a freeze-drying bung into the hole (26), the apparatus may be subjected to a freeze drying procedure.

Alternatively, if the freeze-drying block is pre-frozen before addition of the liquid reagents, the reagents, which are typically in the volumes of 5 μl, freeze within a few seconds of being dispensed.

Devices having freeze dried reagents loaded thereon can then be retrieved by removing the plate (23) and lifting the devices out, if required using an magnetic lifting device.

1. A device for transferring reagents in solid or liquid form from one place to another on an apparatus, said device comprising an upper portion, adapted to be held, and a lower portion adapted to hold reagents in solid or liquid form, externally in an available manner.

2. A device according to claim 1 wherein the lower portion is adapted to carry reagents on an outer surface thereof.

3. A device according to claim 1 wherein the lower portion of the device comprises a detachable open cage structure, adapted to hold solid or liquid reagents.

4. A device according to claim 3 wherein the cage structure is adapted to hold solid reagents.

5. A device according to claim 4 wherein the solid reagents are in the form of beads.

6. A device according to claim 3 wherein the cage structure has one or more apertures in the side-walls thereof.

7. A device according to claim 3 wherein the cage structure is attachable to the upper portion by means of a snap-fit or screw-thread arrangement.

8. A device according to claim 1 in which the said lower portion comprises a wand, having a region or profile in which reagents can be accommodated.

9. A device according to claim 8 wherein the wand is profiled to allow reagents to be accommodated within the profiled regions.

10. A device according to claim 9 wherein the profiled regions comprises dimples or grooves.

11. A device according to claim 9 wherein the profiling is suitably arranged on a lower surface of the wand.

12. A device according to claim 8 wherein the lower surface of the wand is provided with an expanded carrier portion.

13. A device according to claim 12 wherein the expanded portion has substantially hollow or tubular shape.

14. A device according to claim 1 which is at least partially disposable after use.

15. A device according to claim 1 wherein the upper portion of the device is adapted to be held within a transfer means of an assay apparatus.

16. A device according to claim 15 wherein the upper portion is provided with one or more annular flanges, projections or ridges, which allow it to be detachably held within a transfer means of an assay apparatus.

17. A device according to claim 15 wherein the upper portion is provided with a magnet or a ferromagnetic element, allowing it to be held and moved using magnetic transfer means.

18. A device according to claim 17 wherein the upper portion is moulded from plastic blended with iron filings.

19. A device according to claim 17 wherein the upper portion is a “goblet”-shaped reagent carrier where the bowl of the goblet contains a magnetic insert.

20. A device according to claim 1 having reagents loaded externally thereon.

21. A cage structure for use in a device according to claim 3.

22. A reagent loading apparatus comprising a body, provided with holes into which one or more of the devices according to claim 1 may be inserted, such that the lower portion projects out of the body, and into a sealable chamber.

23. Apparatus according to claim 22 wherein the body is a freeze-drying block.

24. An assembly comprising:
(a) a device according to claim 1 and comprising a lower portion; and
(b) a reagent loading apparatus comprising a body, provided with at least one hole into which the device may be inserted such that the lower portion of the device projects out of the body and into a sealable chamber.

25. A method for delivering reagents into a liquid in a reaction vessel, said method comprising introducing a device according to claim 20, into the reaction vessel, and releasing the reagents into the reaction vessel.

26. A method according to claim 25 wherein the reagents are liquids and these are released by shaking the device.

27. A method according to claim 25 wherein reagent release is achieved by at least partially immersing the lower portion of the device into a liquid within the reaction vessel, and allowing the reagents to dissolve or disperse into said liquid.

28. A method according to claim 27 wherein the reagents are PCR reagents and the liquid is a resuspension buffer or an DNA/RNA extract.

29. A method according to claim 27 wherein the device is moved using automatic transfer means.

30. Apparatus comprising a device according to claim 1, and means for transferring said device from a first container to a second container.

31. (canceled)
